(19) United States
(12) Patent Application Publication
(10) Pub. No.: US 2005/0233352 A1
(43) Pub. Date: Oct. 20, 2005

(54) Device and methods for processing samples and detecting analytes of low concentration

(76) Inventor: James Vincent Zoval, Lake Forest, CA (US)

Correspondence Address: Donald Bollella;DB TECHNICAL CONSULTING, 126 Almador, Irvine, CA, 92614, US

(21) Appl. No.: 11/034,227

(22) Filed: Jan. 12, 2005

Related U.S. Application Data
(60) Provisional application No. 60/536,044, filed on Jan. 13, 2004.

(51) Int. Cl. C12Q 1/68; C12M 1/34

(52) U.S. Cl. 435/6; 435/287.2

(57) ABSTRACT

The present invention relates to methods and apparatus for carrying out analysis of a sample and or extraction of an analyte in a sample. More specifically, this invention is directed to methods and apparatus for detection and quantification of bindable substances through affinity reaction with a solid phase linked binding substance or agent. The solid phase is preferably provided by absorbent compressible materials having a high surface to volume ratio such as, for example, a porous compressible material or a bundle of microfibers having one or more binding agents attached thereto. The analyte of interest is captured and carried within the solid phase. Separation of bound analyte from free analytes may be performed by washing the solid phase.

### CROSS REFERENCE TO RELATED APPLICATIONS

The present application claims the benefit of priority from U.S. Provisional Application Ser. No. 60/536,044 filed Jan. 13, 2004 which is incorporated herein by reference in its entirety.

### BACKGROUND OF THE INVENTION

1. Field of invention

The present invention relates to methods and apparatus for extraction, mixing, purification, separation, preparation, reaction, manipulation, and quantitative and qualitative analysis of substances. More specifically, this invention is directed to methods and apparatus for detection and quantification of bindable substances through affinity reaction with a solid phase linked binding agent or substance. The solid phase is preferably provided by materials having a high surface to volume ratio such as, for example, a porous compressible material or a bundle of microfibers having one or more binding agents attached thereto. The analyte of interest is captured and carried within the solid phase. Separation of bound analyte from free analytes may be performed by washing the solid phase.

2. Discussion of the Related Art

The detection and quantification of analytes in the blood or other body fluids are essential for diagnosis of diseases, elucidation of the pathogenesis, and for monitoring the response to drug treatment. Moreover, early detection of low levels of chemical and biological pollutants or analytes of interest such as biochemical agents used in warfare are necessary for determining exposure to such agents and early treatment of exposed individuals to prevent mortality and long term effects from such exposure. Traditionally, diagnostic assays require numerous complicated preparation steps and relatively high concentrations of analyte in a sample. Current methods for analyte specific or semi-specific separation or isolation of chemical or biological species from solution include methods that involve moving the solution over a solid phase with specific binding capabilities. To maximize collection efficiency, the solid phase is typically engineered to have a high surface to volume ratio. A “blot” type capture is an example of this. Since the solution is moved through a porous material with a high surface to volume ratio, efficient capture is achieved. The extracted moieties are bound to the solid phase then the solid phase is washed to remove any non-specifically bound species. Another method of analyte specific separation or isolation is done through the use of magnetic particles. Magnetic particles coated with specific binding agents or moieties are mixed in a solution having an analyte of interest. The analytes then bind with the binding agents and the magnetic particles with the extracted analytes or moieties are removed from the solution using a magnet. Then the solid phase (magnetic particles) is rinsed to remove any non-specifically bound species. These assays require relatively high concentrations of analyte in a sample and numerous complicated preparation steps which are labor intensive and require numerous pipetting steps. Thus, there is a significant need for devices and methods for fast and efficient detection and quantitation of analytes of low concentration requiring less sample manipulation.

### SUMMARY OF THE INVENTION

Analysis of samples aimed at the quantitative and qualitative determination of substances associated with biochemical warfare, physiological disorders, biomedical research, proteomics, environmental studies, agriculture, and food industry, relies on chemical test and specific binding assays from which the immunoassays and genetic tests play a dominant role. The outstanding specificity and sensitivity for qualitative and quantitative determination of an almost limitless number of analytes in practically any milieu, and the ability to miniaturize and adapt to automation makes them ideal tools for routine assays.

Antibody binding techniques are based on the interaction of a binding antibody, receptor, or other binding proteins with an antigen or a specific ligand molecule and the formation of an antibody-antigen or receptor-ligand complex. By changing certain conditions a binding assay can be designed to determine an analyte, ligand, or target binding reagent or an antibody of interest. The steps are similar but the assay configuration provides results pertinent to the antigen or antibody of interest. Similarly, genetic assays are based on the interaction and binding of specific complementary sequences of DNA and or RNA.

One aspect of the present invention includes a solid phase for sample extraction. The solid phase may be a “sponge” or a “mop” with analyte selective binding capabilities expressed throughout. The sponge may be formed from a porous compressible material that adsorbs liquid. When the material is saturated or semi saturated with liquid it expels that liquid when compressed to a smaller volume. The mop may be formed from fibers that, as a bundle, can adsorb liquid. When the fiber bundle is saturated or semi saturated with liquid it expels the liquid when compressed to a smaller volume. The compressible material may be made by extrusion molding methods of open cell material making some surfaces closed cells. The compressible material and its solid support may be arrayed in 2 or 3 dimensions.

The sponge is preferably formed from Polyvinyl Alcohol or PVA. PVA possesses a three dimensional open cell structure similar to that of natural sea sponges. All of its cells are interconnected, not independent, i.e., open pore. Major advantages of this physical structure are its high filtering efficiency, its ability to be reused after cleaning, and its favorable retention and wicking properties. A PVA sponge will absorb up to 12 times its dry weight in water. When saturated with water, it becomes flexible and soft like natural sea sponge. The wet volume is about 20% greater than the dry volume. PVA exhibits mechanical strength and abrasion resistance equal to or greater than any other synthetic sponge material. Pore size and shape can vary to meet specific applications. Wet PVA sponge will withstand temperatures to 90 degrees C. without deformation. PVA is normally pure white. It can, however, be pigmented in any color and to a high degree of color-fastness.

During the manufacture of a PVA sponge, a water-soluble porous structure is chemically insolubilized. The material will withstand the action of dilute acids, strong alkalis, and solutions of common detergents. Some detergents of the sulfonate category (over 5% strength) will slowly swell and weaken the sponge. Organic solvents do not, as a rule, affect the sponge unless they are water-miscible and are applied mixed with 30% to 60% water. In that case the sponge will swell and be weakened. Thorough washing in water will return the sponge to its original state. PVA is also not compatible with nickel sulphate solutions. PVA sponge behaves in water as a negatively charged colloid and will strongly adsorb metallic cations such as copper or iron. It may act like an ion exchange resin in this respect. It also has strong affinity for cationically charged organic ions of the quaternary ammonium type. PVA sponge, itself, normally does not support the growth of bacteria or molds, nor will it destroy those organisms. PVA foam packaged wet should preferably be treated chemically to inhibit bacteria or mold growth. Rust stains on PVA may be removed in the same way; as they are from cotton using a solution of oxalic acid, or citric or tartaric acid. Furthermore, sodium hypochlorite solution degrades the sponge.

Another aspect of the present invention is the use of the porous compressible material or sponge to extract and mix solutions within the sponge to increase the rate of a reaction and prevent a concentration gradient from forming during a chemical reaction. This increases the efficiency of the reaction and decreases the time needed for the reaction to come to completion thereby allowing the user to get results faster and allowing more tests to be run at any given time.

One embodiment of the present invention is a sample processing apparatus for absorbing or contacting a sample having a sample loading vessel for containing the sample; and a porous compressible material that is placed in the sample loading vessel wherein it absorbs a portion or the entire sample and incorporates one or more analytes of interest in the sample. The sample processing apparatus may also include a means to compress the compressible material. When the compressible material is compressed it preferably excludes the sample. Repeated expansion and compression of the compressible material causes the sample to flow in and out of the compressible material thereby aiding in mixing of the sample. The sample processing apparatus may further include a plunger attached to a surface or portion of the compressible material to aid in compression, transportation, manipulation, and manual handling of the compressible material.

The processing apparatus may also have a means for connecting the plunger to a translation device capable of moving the plunger 1 to 3 dimensions. The sample in the compressible material may be transferred into a collection vessel having a grid. The sample is displaced from the compressible material by compressing the material against the grid causing liquid to be expelled into the collection vessel. The sample may also be expelled from the compressible material by compressing the material on the side of the collection vessel or a solid portion of the collection vessel.

The present invention is further directed to an apparatus including a member for compressing the compressible material against the surface or grid positioned within a collection vessel allowing extruded liquid/solution/mixture to be displaced and collected away from the material. This apparatus is further provided with a member for moving the compressible material to the collection vessel so that the material is compressed against the surface or grid positioned in the collection vessel allowing extruded liquid/solution/mixture to be displaced and collected away from the material.

Alternatively the compressible material may be compressed against an absorbent material such as a membrane causing the sample to be expelled from the compressible material and transferred into the absorbent material.

The compressible material may be formed such that it can adsorb aliquots of sample and extrude aliquots into one or more collection vessels by the compression described in above. The compressible material may be made to selectively adsorb or bind one or more targets or analytes or one or more classes of analytes. The analytes may include chemical substances and biological materials such as cells, colloids, particles, tissues, sub-cellular components, genetic material, proteins, and antibodies.

In another embodiment of the present invention the compressible material is treated or chemically modified to selectively adsorb or bind a single analyte or class of analytes. The chemical modification can be made to some or all areas of compressible material. Binding agents may be attached to the surface of the compressible material by, for example, adsorbing antibodies, chemically bonding antibodies, silanating organic polymer for DNA or RNA binding, adsorbing or binding DNA, and any technique for attaching molecules onto a surface know in the art may be used in conjunction with the present invention.

In yet another embodiment of the present invention, particles that have properties for selective adsorption or binding analytes of interest may be embedded within the pores of the compressible material. The particles may include, for example, silica, plastic, or metal particles having antibodies, antigens, or genetic material (oligonucleotides) attached thereto and metal particles for chelating charged molecules. This embodiment may include an element for embedding the particles in the compressible material; an element for attaching particles to the compressible material after it has been manufactured; and an element for treating or modifying the compressible material.

The sample processing apparatus of the present invention may include a device that executes repeated compression and decompression of the compressible material to effect mixing and allow maximum exposure of the sample to binding surface of the compressible material and to the binding agents attached thereto.

The sample processing apparatus of the present invention may also include a device that executes one or more rinses of the compressible material to remove non-specifically bound moieties by compressing and decompressing the compressible material in a vessel containing a rinse solution followed by permanent extrusion of rinse solution. This entire rinsing procedure can be repeated as needed in a vessel with fresh rinse solution.

Further aspects of the sample processing apparatus of the present invention includes removing specifically bound analytes or moieties from the compressible material and collected the analytes in a vessel; a means for removing the analytes; a means for detecting, identifying, and quantifying the analytes. Analytes may include, for example, DNA, RNA, proteins, antibodies, small molecules, cells, cellular components, and antigens. The DNA may be amplified on the compressible material.

The apparatus may have a liquid output channel on bottom of the vessels where extruded liquid can be removed from contact with compressible material and include a means to exert force on fluid in the compressible material for removal from any of the vessels through the output channel. The force may be caused by vacuum, gravity ,or centrifugal force.

Yet another aspect of the apparatus of the present invention includes cleavable subunit connecting the compressible material and the binding agent. The cleavable subunit may be cleaved chemically, enzymatically, thermally, mechanically, or photometrically (UV).

Still another aspect of the apparatus involves a signal agent contacted and mixed as described earlier by repeated compression and decompression of the compressible material containing the specifically bound analytes. The signal agent may be attached to a reporter for detection. The reporter may be an enzyme, fluophore, chromophore, dye, radioisotope, or any detectable substance. The apparatus then may have detection capabilities for detecting one or all of the following: absorption, fluorescence, luminescence, and radioisotope detection.

The apparatus of the present invention may include capabilities for controlled heating and cooling of the vessels or compressible material and have digital or analog outputs for communication with external peripherals such as data processing systems.

The compressible material of the present invention may also be used for mixing reagents and samples by repeated compression and decompression of the compressible material thereby increasing the rate of the reaction between analytes in the sample and reagents. This may significantly decrease the reaction time of a test. Repeated compression and decompression of the compressible material also lessens the time required for sample binding to a solid phase for binding assays, discussed above, relative to passive diffusion based binding assays, or single pass chromatographic assays. This mixing method of the present invention is also advantageous in comparison to relatively slower mixing and analyte capture using magnetic particles on rotisserie racks.

Sample Application and Analyte Capture

When a sample is placed in the sample loading vessel and the sample absorbed into the sponge or mop solid support having a binding agent or capture probe attached thereto, the analyte including, for example, target antigen or antibody, present in the sample binds to the binding agent on the solid support. The binding agent may be an antigen recognized by an antibody analyte or an antibody or receptor with specific affinity to the target antigen or ligand (analyte). Following the binding step, unbound analyte is removed through a wash step. It should be understood that various techniques, procedures, and chemistries, know in the art, may be used to bind the binding agent onto the solid support. These include, but are not limited to, direct covalent binding of probes onto a chemically activated surface, passive adsorption, and through cross-linking reagents.

In addition to surface chemistries for attaching binding agents or capture probes, blocking agents may be used to block areas within the solid support where capture probes are not bound (non-capture areas) to prevent non-specific binding of the target or analyte, signal probes, and reporters onto these areas. Blocking agents include, but are not limited to, proteins such as BSA, gelatin, sugars such as sucrose, detergents such as tween-20, genetic material such as sheared salmon sperm DNA, and polyvinyl alcohol.

Signal Generation

Signal is generated from tags or labels attached to signal or reporter agents or probes that have specific affinity to the analyte bound to the binding agents on the solid support. Signal agents or probes may include, for example, signal antibodies or signal ligands, tagged with fluorescent, phosphorescent, luminescent, or chemiluminescent molecules and enzymes. The enzymes may facilitate a chemical reaction that produces fluorescence, color, or a detectable signal in the presence of a suitable substrate. For example, conjugated horseradish peroxidase (HRP; Pierce, Rockford, Ill.) may be used with the substrate 3,3,5,5-tetramethylbenzidine (TMB; Calbiochem cat. no. 613548, CAS-54827-17-7) in the presence of hydrogen peroxide to produce an insoluble precipitate. Horseradish peroxidase (HRP) can also be used in conjunction with CN/DAB (4-chloronaphthol/3,3′-diaminobenzidine, tetrahydrochloride), 4-CN (4-chloro-1-napthol), AEC (3-amino-9-ethyl carbazol) and DAB (3,3-diaminobenzidine tetrahydrochloride) to form insoluble precipitates or it may be used with ABTS [2,2′-azino-bis(3-ethylbenzthiazoline-6-sulfonic acid)] or TMB to produce a change in color of the substrate that may be measured using a UV-Vis Spectrophotometer at 405 nm wavelength. Similarly, the enzyme alkaline phosphatase (AP) can be used with p-nitophenyl phosphate to produce a product detectable at 405 nm or 5-bromo, 4-chloro,3-indolylphosphate (BCIP)/nitroblue tetrazolium (NBT) in the practice of the present invention. Other suitable enzyme/substrate combinations such as those used in micro well applications may be used in conjunction with the present invention as would be apparent to those of skill in the art.

Detection

The signal generated by the signal agents or the enzyme reaction can be detected and quantified using a suitable detection apparatus further described below.

### BRIEF DESCRIPTION OF THE DRAWING FIGURES

Further objects of the present invention together with additional features contributing thereto and advantages accruing therefrom will be apparent from the following description of the preferred embodiments of the invention which are shown in the accompanying drawing figures with like reference numerals indicating like components throughout, wherein:

FIG. 1A is an illustration of a compressible material having interconnected pores or cells;

FIG. 1B is a perspective see-through view of the compressible material of FIG. 1A showing the connections between the pores;

FIG. 2 is a perspective view of the compressible material having multiple pores of different sizes and shapes;

FIG. 3A is a pictorial representation of the compressible material attached to a handle or plunger;

FIG. 3B is a depiction of the compressible material of FIG. 3A being compressed by the plunger;

FIG. 4 is a pictorial illustration of a method for transferring fluids using the compressible material of the present invention;

FIG. 5 shows a method for mixing and transferring a solution using the compressible material;

FIG. 6 shows steps of a method for processing samples in an assay;

FIG. 7 is a perspective view of different vessels that may be used for sample processing;

FIG. 8 is an illustration of an apparatus for sample processing;

FIGS. 9A to 9D depict a method for detecting an analyte using the compressible material in an immunoassay implementation of the present invention;

FIGS. 10A to 10D show steps of a method for detecting an oligonucleotide sequence of interest in a sample using the compressible material in a genetic assay implementation of the present invention;

FIG. 11A is a perspective view of an alternate embodiment of the present invention using a microfiber material as a solid phase for sample extraction and manipulation;

FIG. 11B is a perspective view of the microfibers compressed on a solid platform;

FIGS. 12A to 12D illustrate a method for detecting an analyte using the microfiber material in an immunoassay implementation of the present invention;

FIGS. 13A to 13D represent steps of a method for detecting an oligonucleotide sequence of interest in a sample using the microfiber material in a genetic assay implementation of the present invention; and

FIG. 14 shows release of captured genetic material and its signal probe from capture probes bound to the microfiber material.

### DETAILED DESCRIPTION OF THE INVENTION

The present invention is directed to a sample processing and analysis apparatus. It is further directed to binding assays, including for example immunoassays and genetic assays, and related detection methods. Each of these aspects of the present invention is discussed below in further detail.

Immunoassays

There are three classes of binding assays. These include binding protein capture assays, analyte capture assays, and sandwich type assays. The latter assay type can have a binding protein-analyte-binding protein or analyte-binding protein-analyte format.

A specific implementation of a binding assay is an immunoassay. In such an immunoassay, the binding protein may be represented by a capture antibody or a capture antigen and the analyte may be an antigen/hapten or a target antibody, respectively. The product of the reaction is an antigen-antibody immune complex.

Quantification of antigen molecules is most efficiently done by a two-antibody sandwich assay. The capture antibody is immobilized on the solid support and the signal antibody is tagged or labeled with a suitable reporter. The recognition of the same antigen by two different binding antibodies, namely the solid phase capture antibody and the reporter linked signal or enumerating antibody, contributes to the exquisite specificity of the assay. The capture antibody identifies a first epitope on the surface of the analyte molecule while reporter or signal antibody recognizes a second epitope at a different location on the surface of the same analyte molecule. The signal generated by the capture antibody-antigen-signal antibody complex is proportional to the amount of the bridging analyte present in the sample. The concentration of antigen in the analyzed specimen can then be determined through comparison with the signal generated by known quantity of pure antigen.

Detection or quantification of an antibody or any immunoglobulin is alternatively done by a solid phase immobilized antigen test device. The analyte or target antibody is allowed to bind to the capture antigen creating an immobilized antigen-antibody complex. A labeled form of an anti-immunoglobulin antibody or other immunoglobulin specific binding protein such as protein A and protein G, is then applied to the immobilized antigen-antibody complex which enumerates the analyte antibody through binding of the signal antibody to a site other than the epitope binding site of the target antibody. Detection of the signal generated directly or indirectly by the tagged reporter or signal antibody becomes a measure for the presence and quantity of the analyte antibody when comparison with a known reference material for the immunoglobulin is established.

More recently, antibodies are determined by antigen sandwich, dubbed “inverse sandwich” immunoassays. This assay makes use of the presence of two equal epitope binding sites on each immunoglobulin G (IgG) molecule, thus allowing for a simultaneous binding of the analyte antibody to two separate antigens, solid phase bound capture antigen and reporter antigen. Reporter represents the labeled form of capture antigen. Lateral flow antigen sandwich immunoassays have one antigen/hapten immobilized to a solid phase, most frequently a nitrocellulose or nylon membrane, and the second antigen, carrying the same epitope as the solid phase bound antigen, labeled with enzyme, radioisotope, dye, or other signal generating substance. Antibody specific to the epitope represented by both antigens can than be specifically detected in a single step assay procedure.

Genetic Assays

The present invention is also directed to the detection and analysis of target nucleic acid sequences present in test samples. Target nucleic acids suitable for use with the present invention include both deoxyribonucleic acid (DNA) and ribonucleic acid (RNA), including mRNA, rRNA, hnRNA, siRNA and tRNA.

Target nucleic acid may be used directly from a biological sample or amplified prior to testing via polymerase chain reaction (PCR) or isothermal amplification to generate amplicons. If using PCR for amplification, RNA may first be reverse transcribed into DNA using techniques well known in the art. Target nucleic acid may be single stranded or double stranded. If double stranded, the nucleic acid may be denatured prior to hybridization with capture DNA.

The present invention may be used to detect specific nucleic acid sequences in a wide variety of biological samples, including but not limited to bodily fluids such as whole blood, serum, plasma, saliva, urine, lymph, spinal fluid, tears, mucous, semen and the like, agricultural products, food items, waste products, environmental samples, such as soil and water samples, or any other sample containing, or suspected of containing specific nucleic acid sequences of interest. For example, the present invention may be used to detect the presence of particular strains of microorganisms, such as viruses or bacteria, in body fluids or environmental samples, by detecting the presence of particular nucleic acid sequences in the sample. Other uses of the present invention will be apparent to those of skill in the art given the present disclosure.

Capture DNA oligonucleotides, or probes, are immobilized onto the surface of the solid support as described below. Target DNA or RNA is then hybridized on the capture probes to thereby “capture” the target nucleic acid in the solid support for further processing and detection. The sequence of the capture DNA is selected so as to hybridize directly with target DNA or RNA, thereby forming a complex including capture DNA, target DNA, or RNA

It is thus the aim of the present invention to process and analyze samples for all antibody and antigen binding assays including cell related assays, and probe assays from micro-titer plate, test tube, gel, membrane, or glass slide format and genetic assays using the compressible material or the microfiber embodiments of the apparatus of then present invention. Furthermore, multiple and lengthy incubation steps, washing steps, reagent addition steps and similar processing steps are reduced.

Linking Binding Agents onto Solid Support

Attachment of the binding agent or capture probe to the solid support may be achieved using cross-linking agents. Cross-linking agents include, but are not limited to homobifunctional linkers, heterobifunctional linkers, and zero-length cross-linkers. Homobifunctional linkers are linkers with two reactive sites of the same functionality, such as glutaraldehyde. These reagents could tie one protein to another by covalently reacting with the same common groups on both molecules. Heterobifunctional conjugation reagents contain two different reactive groups that can couple to two different functional targets on proteins and other macromolecules. For example, one part of a cross-linker may contain an amine-reactive group, while another portion may consist of a sulfhydryl-reactive group. The result is the ability to direct the cross-linking reaction to selected parts of target molecules, thus garnering better control over the conjugation process. Zero-length cross-linkers mediate the conjugation of two molecules by forming a bond containing no additional atoms. Thus, one atom of a molecule is covalently attached to an atom of a second molecule with no intervening linker or spacer. Implementations of the embodiments of the present invention utilize binding or capture agents to perform the assays described herein. It should be understood that a capture or binding agent refers to any macromolecule for detecting an analyte. The capture agents of the invention include macromolecules preferentially selective, or having a selective binding affinity, for an analyte of interest. Capture agents include, but are not limited to, synthetic or biologically produced nucleic acid and synthetic or biologically produced proteins. Examples of capture agents that can be employed by this invention, include, but are not restricted to, deoxyribonucleic acid (DNA), ribonucleic acid (RNA), oligonucleotides, polymerase chain reaction products, or a combination of these nucleotides (chimera), antibodies (monoclonal or polyclonal), cell membrane receptors, and anti-sera reactive with specific antigenic determinants (such as on viruses, cells, or other materials), drugs, peptides, co-factors, lectins, polysaccharides, cells, cellular membranes, and organelles. Antibodies include, but are not limited to, polyclonal, monoclonal, and recombinantly created antibodies. Antibodies of the invention can be produced in vivo or in vitro. Antibodies of the invention are not meant to be limited to antibodies of any one particular species; for example, antibodies of humans, mice, rats, and goats are all contemplated by the invention.

From the many known analytical and biochemical methods, the most widely used procedures for quantitative and qualitative analysis of complex samples are protein binding assays and genetic assays based on selective affinity of the capture agent or binding reagent and the analyte as described above.

Passive adsorption is one preferred method for achieving the linkage of a bio-chemical, chemical, or other binding reagent to a solid support. Large bio-molecules containing pockets of hydrophobic amino acids, carbohydrates, and similar components are easily linked to a non-polar surface through passive adsorption. The hydrophobic forces exhibited by the solid support and the bio-molecule, as well as the electrostatic interaction between the solid support and the bio-molecule, result in the formation of a stable linkage. The pH, salt concentration, and presence of competing substances will, among other factors, determine the extent to which various binding proteins link non-covalently to the plain surface of the solid support. Another critical aspect of immobilizing binding proteins or capture agents onto a solid support is the retention of functional activity of the capture or binding agent. Frequently, protein capture agents loose their biochemical properties due to denaturation in the process of immobilization involving structural reorganization followed by conformational changes and accompanying changes of functionally active sites. Enzymes, receptors, lectins, and antibodies are examples of such bio-polymers, binding proteins, or capture agents.

Situations where the lack of passive interaction with the solid support or the loss of functional activity due to the immobilization process, necessitate another approach. The approach taken in these cases leads to the functionalization of the surface of the solid support upon which the immobilization of the biochemical reagent is intended. Functionalization is a process by which the solid support surface is modified by attaching specific molecules or polymers with functional groups to the surface. The functional groups are then used to bind recognition molecules such as binding proteins, capture antibodies, receptors, DNA probes, RNA probes, and other similar assay components.

Chemical modification of the surface of the solid support is efficiently done through grafting procedures that allow the deposition of a thin interphase layer, active layer, or interlayer on the solid support. Ideally, the interphase layer should make a stable linkage of the grafted material to the substrate surface and contain a spacer molecule ending in a functional group or variety of chemically different functional groups. This allows the selection of specific surface chemistries for efficient covalent immobilization of a variety of capture agents with different demand for spatial orientation, side directed attachment within the structure of the binding agent. The introduction of spacer molecules contributes significantly to the flexibility and accessibility of the immobilized binding or capture agents. By placing a spacer layer between the solid phase of the solid support modified or grafted with different functional groups and the binding agent, a potentially denaturing effect of the direct contact of the binding with the functional groups is eliminated.

Selective binding tailored chemistries permit the retention of functional activity of the immobilized capture molecule or agent. As a consequence, one can expect chemistries on the solid phase/liquid phase interphase of the capture agent-analyte to approach those of the liquid phase. This is especially true with the increased access of the analyte as processed in the compressible solid support and microfiber material, for example. A potential benefit of a graft modified substrate surface is the “normalization” of the surface with respect to the uniformity in density of the immobilized binding protein. Also, bonds between capture reagent and graft mediated solid support become more uniform. This results in holding each molecule of binding protein with the same bond energy. This aspect becomes of paramount importance for any quantitative assay especially on the design of protein and DNA assays.

Compressible Solid Support

As discussed above, one embodiment of the present invention includes an open pore compressible solid support such as a sponge-like material. The compressible solid support is preferably formed from a matrix of cross-linked poly(vinyl) alcohol (PVA) having open pores or interconnected cells or ports similar to sea sponges. FIG. 1A is an illustration of the compressible material 100, also herein referred to as sponge, having interconnected pores or ports 102. These pores are connected through channels 104, shown in FIG. 1B, formed from the interconnections between the cells 102 within the compressible material 100 as in sea sponges. The pores 102 may be uniform in size and shape or may vary depending on the polymerization process. A compressible material 100 having multiple interconnected pores 102 of different sizes and shapes is depicted in FIG. 2. This compressible material may look like a common household sponge. The use of PVA to form compressible materials is known in the art. The various techniques of polymerization to form different pore or cell sizes are also know in the art.

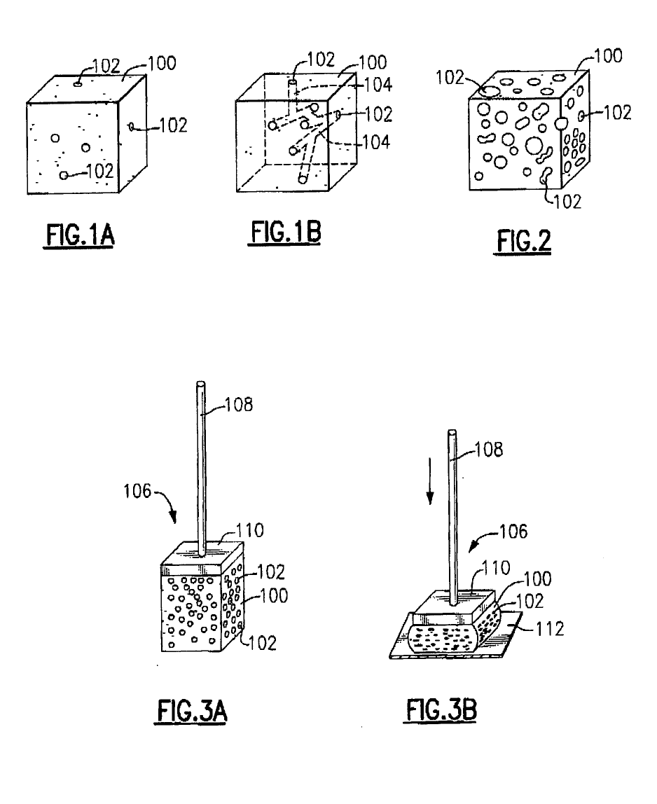

The compressible material 100 of the present invention may be attached to a plunger 106 having a handle 108 and a base 110. The sponge 100 is connected at the base of the plunger as illustrated in FIG. 3A. The plunger is used to manipulate the sponge 100 and aid in ease of its compression and decompression. FIG. 3B next shows the sponge 100 being compressed on a solid platform 112 using the plunger 106. As illustrated, the pores or cells 102 are also compressed during this process. A main characteristic of a sponge is its ability to absorb liquid and expel same upon compression.

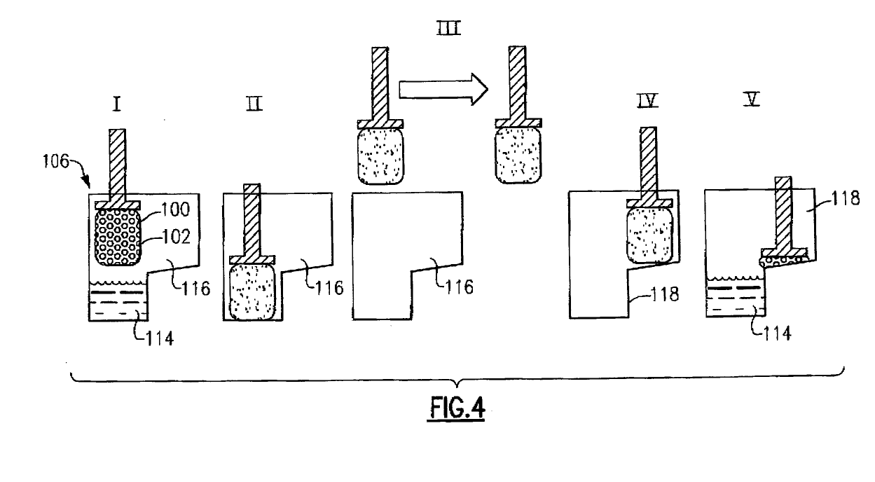

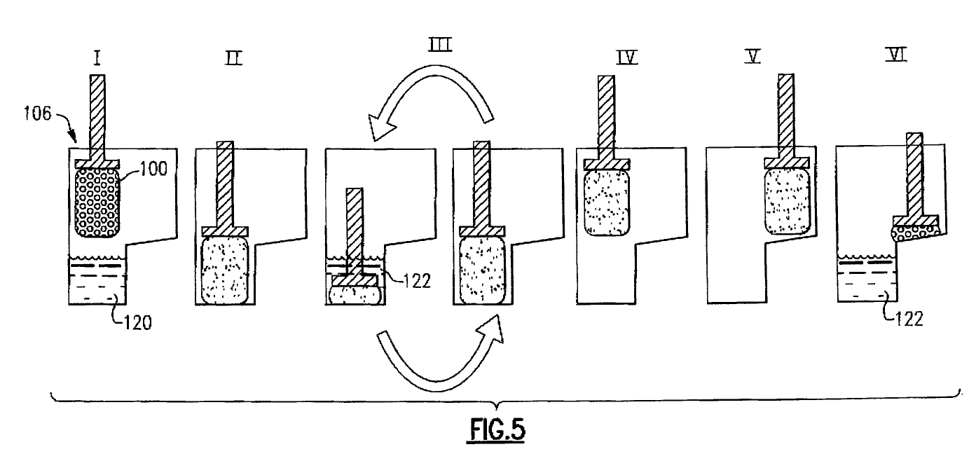

Referring next to FIG. 4, there is depicted the use of the sponge 100 of the present invention to collect and transfer a liquid, solution or mixture 114 from one vessel to another. As shown in Step I of FIG. 4, liquid 114 in vessel 116 is placed in contact with the sponge 100. The liquid is then absorbed by the sponge 100, Step II. The liquid in the sponge may then be transported to another vessel 118, Steps III and IV. The liquid 114 may then be released in to vessel 118 by compressing the sponge 100 as shown in Step V.

The sponge or compressible material of the present invention may be used to mix then transport a solution as depicted in FIG. 5. During Steps I and II, the solution 120 is absorbed into the sponge 100. Solution 120 is then mixed in Step III by repeated compression and decompression of the sponge 100. The mixed liquid 122 is then transported into another vessel, Steps IV to VI.

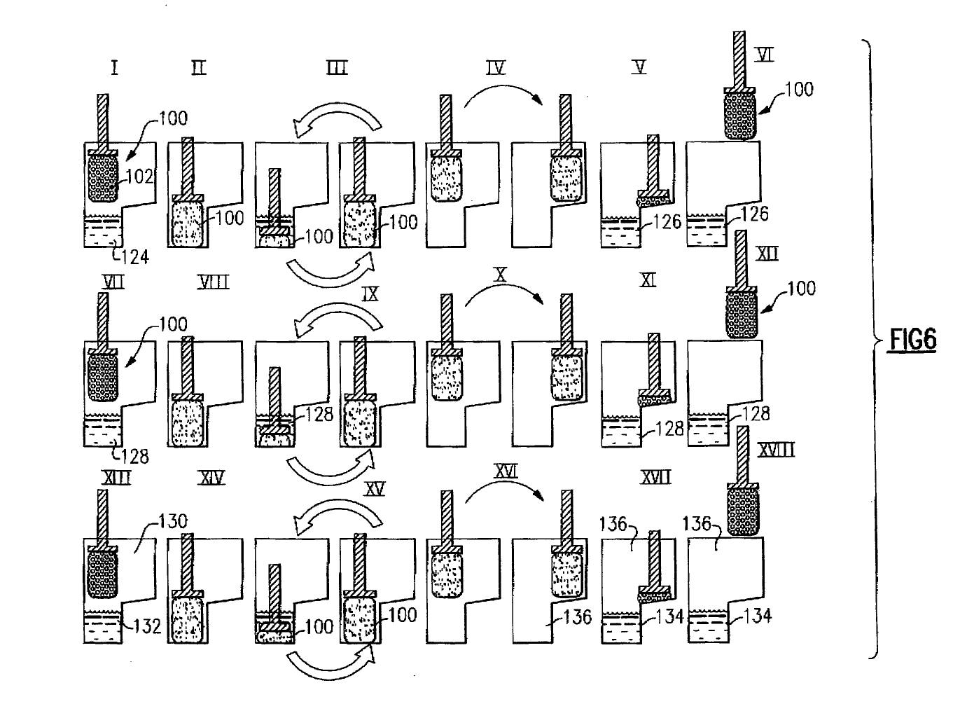

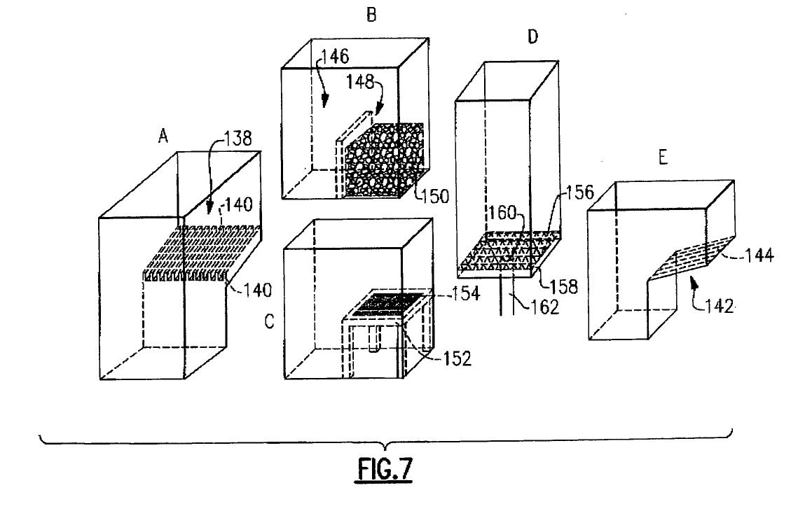

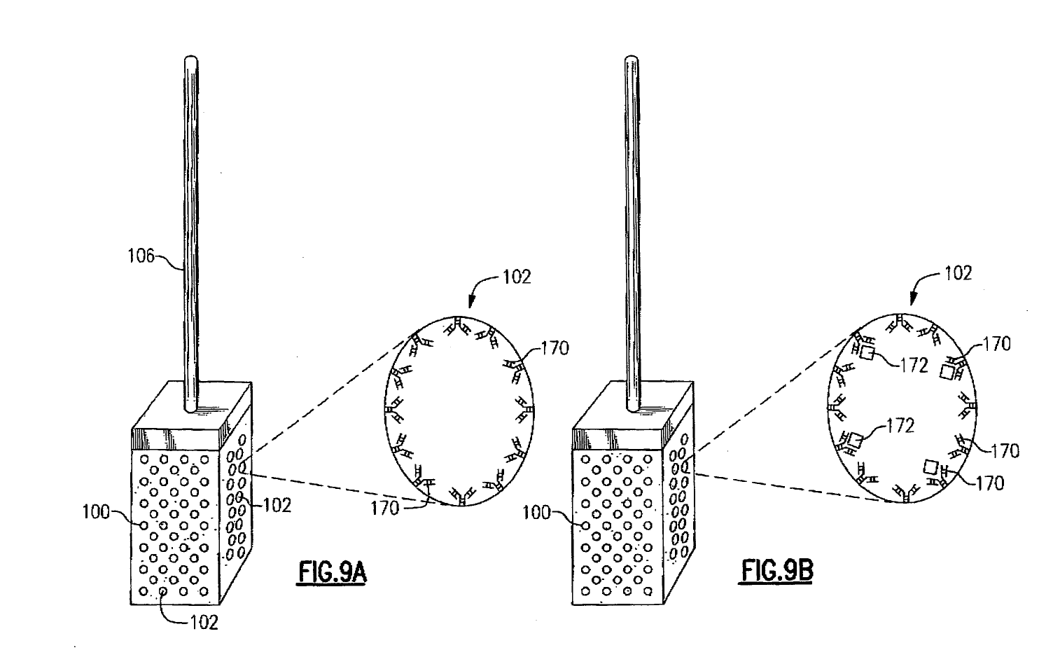

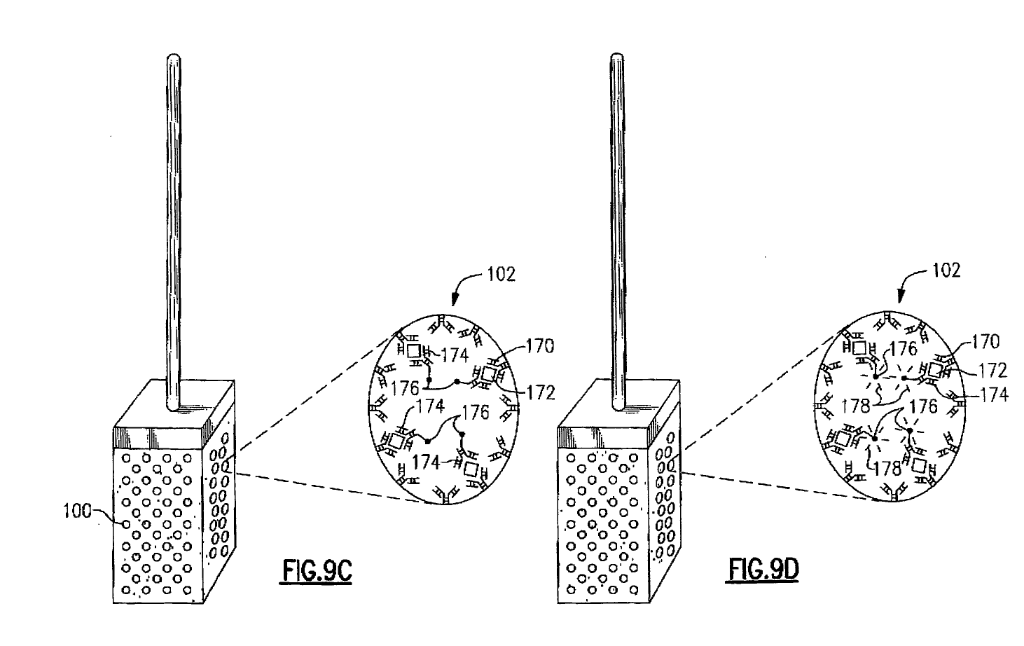

Turning now to FIG. 6, there is illustrated a method for isolating a sample including mixing, binding, transport, washing, and analyte isolation steps. The first step in this method is the absorption of a sample solution 124 containing at least one analyte of interest into the sponge 100 in Steps I and II. Binding agents are attached to the surface of the interconnected cells using the techniques described above or any appropriate conjugation process known in the art. Binding agents may include antibodies, antigens, genetic material, and any molecule capable of binding or capturing an analyte of interest. In Steps II and III, analytes in the sample 124 are exposed to the binding agents on the cells 102. Incubation time for analyte binding onto the binding agents is decreased by repeated compression and decompression of the sponge 100, Step III. This process enhances the kinetics of analyte-binding agent interaction by allowing multiple passes of the analyte over the capture area or surface of the interconnected cells of the sponge 100. After the active incubation step (Step III), the original buffer 126 from the sample solution 124 is discarded in Steps IV and V. The sponge 100 containing bound analyte is then washed to remove non-specifically bound analyte or other non-specific contaminants present on the capture area by placing the sponge 100 in a wash buffer 128; Steps VII and VIII. The sponge 100 is compressed and decompressed repeatedly in Step IX to facilitate removal of non-specifically bound contaminants. After the washing step, the wash buffer 128 is then squeezed out of the sponge; Steps X, XI and XII. The sponge is then moved to an analyte collection vessel 130 containing elution reagents 132 that facilitate removal of the analyte from the binding agents; Steps XIII and XIV. Alternatively, such as in DNA assays, an analyte such as DNA may be released from its complementary DNA probe by heating the sponge to around 90 degrees C. to remove the analyte DNA. The elution buffer may also be heated to 90 C to further enhance removal of the DNA analyte. The removal of the analytes is facilitated, in Step XV, by repeated compression and decompression of the sponge 100. The resulting isolate solution 134 containing the analyte of interest is then released into a collection vessel 136; Steps XVI, XVII, and XVIII. The isolate may then be stored for further use. The binding agents, analyte binding and analysis are best shown and described below in conjunction with FIGS. 9 to 14.

Next in FIG. 7, there are shown various designs of vessels that may be used for the sample processing method described in above in conjunction with FIG. 6. These vessels facilitate the release of liquids from the sponge. Vessel A includes a corrugated ledge 138 which allows fluid from the sponge to flow through its channels 140 when the sponge is compressed on the ledge 138. Similarly, Vessel E has a ledge 142 having channels 144 that aid in efficient release of fluids from the sponge 100. Vessel B includes two wells, one open well 146 for liquid containment and fluid absorption well 148 containing a fluid absorbing material 150. Well 146 may be filled with a wash buffer where the sponge containing bound analyte may be washed as described above in conjunction with FIG. 6. The wash buffer may then be discarded from the sponge 100 by releasing it into the fluid absorbing material 150 by compressing the sponge on material 150 thereby releasing the fluid in the sponge into material 150. The next vessel, Vessel C, has a mesh 152 on a raised platform 154. Fluid is released from the sponge by compressing the sponge on the mesh 152. Vessel D includes a mesh 156 above its base 158, an exit port 160 and a drain tube 162. Fluid may be released from the sponge by compressing the sponge on mesh 156 and removal of the fluid is facilitated by applying a vacuum though the drain tube 162.

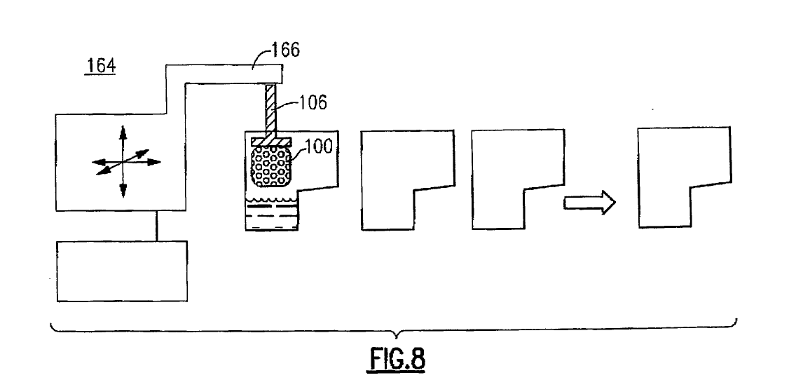

Turning now to FIG. 8, there is shown a sample processing apparatus 164 that may be used in conjunction with the present invention. Processing of samples as described above in conjunction with FIGS. 6 and 7 and below in conjunction with FIGS. 9 to 14, may be done using the sample processing apparatus 164. Apparatus 164 may be attached to the plunger 106 through an arm 166 and automatically carry out pre-determined sample processing steps such as sample mixing, transport, washing, elution, and analysis steps. The apparatus preferably is capable of moving the arm 166 in three dimensions.

FIGS. 9A to 9D next illustrate a method for detecting an analyte using the compressible material in an immunoassay or binding assay implementation of the present invention. Binding assays, as described above, may use antibodies as binding agents. The assay described in conjunction with FIGS. 9A to 9D is an antibody-analyte-antibody sandwich assay. As would be apparent to one of skill in the art, the use of the compressible material of the present invention is not limited to the antibody-analyte-antibody sandwich assay but may be used for all binding assays described above. In FIG. 9A, there is illustrated a magnified view of a cell 102 from sponge 100. As shown, capture antibodies 170 (the binding agent) are attached or conjugated on the surface of the cell 102. Sponge 100 may then be exposed to a solution containing an analyte 172 as best described above in Steps I-VI of FIG. 6. The sponge may then be washed to remove non-specifically bound contaminants as described above in conjunction with Steps VII-XII of FIG. 6. FIG. 9B shows analyte 172 bound to the capture agent 170. The sponge is then immersed and incubated in a solution containing signal agents or signal antibodies 174, as in Steps I-VI in FIG. 6, having attached thereto a signal element 176 such as a detectable label such as a fluorescent label or a catalyst or enzyme capable of producing a detectable signal such as, for example, the enzymes described above including horse radish peroxidase (HRP). The sponge is once again washed with a wash buffer following the steps described above in conjunction with FIG. 6, Steps VII-XII. FIG. 9C depicts signal agents 174 having a signal element 176 bound to the analyte 172. If the signal element is an enzyme capable of producing a detectable signal such as HRP, the sponge is then immersed in a reagent solution containing an enzyme substrate, as in Steps XIII-XV. In the case of HRP, the enzyme substrate may be TMB or ABTS. The enzyme substrate then reacts with the signal element producing a detectable signal 178 as illustrated in FIG. 9D. The solution containing the detectable product 178 is then released into an analysis tube as in Steps XVI to XVIII of FIG. 6. The amount of product 178 is then quantitated using an appropriate analytical instrument. If HRP/ABTS is used to produce the detectable signal 178, the resulting product may be quantitated using a UV-Vis Spectrophotometer.

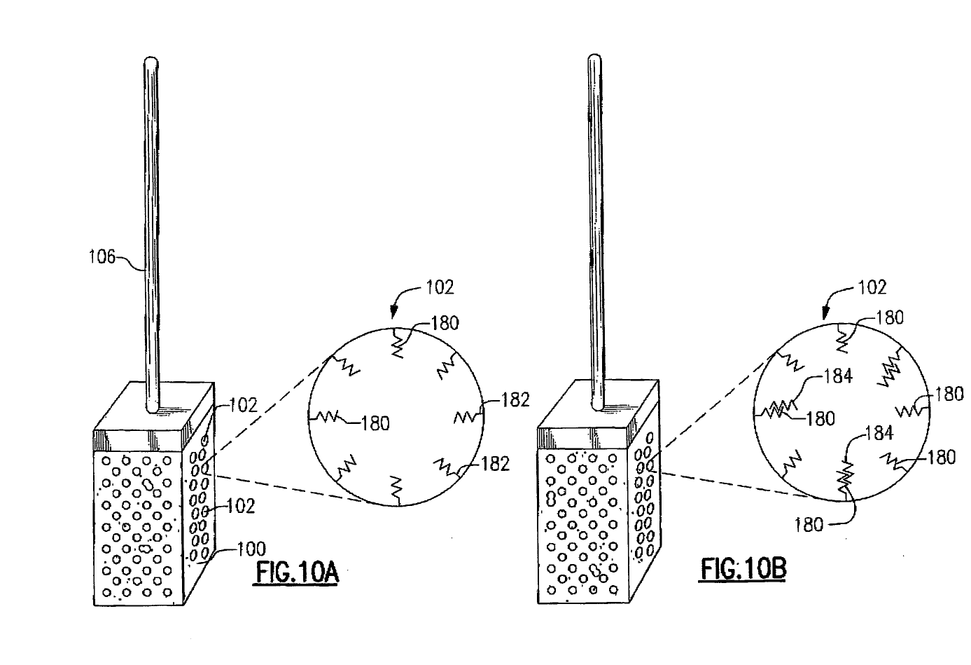

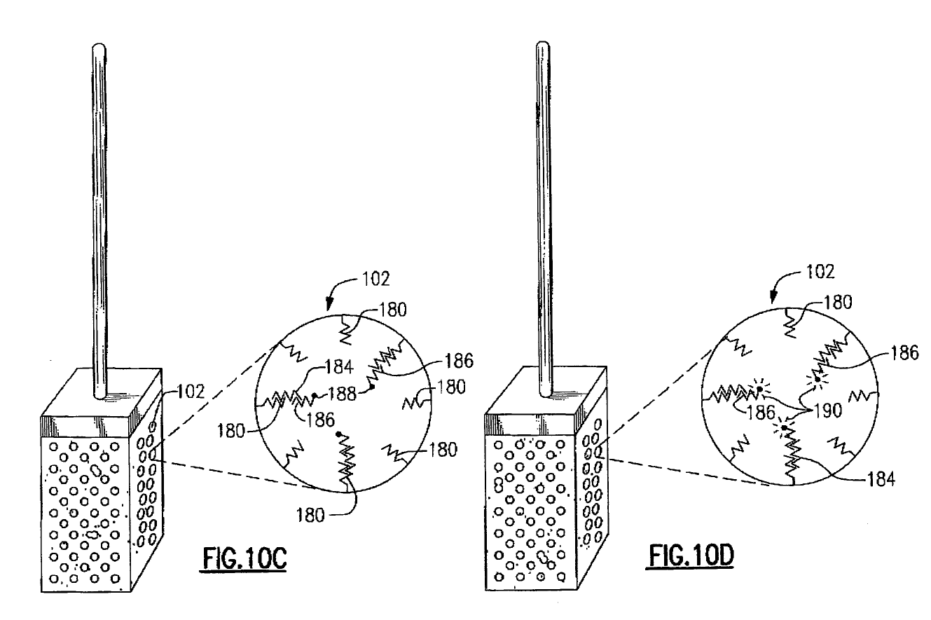

With reference next to FIGS. 10A to 10D there are shown steps for detecting an oligonucleotide sequence of interest in a sample using the compressible material in a genetic assay implementation of the present invention. In FIG. 10A, there is illustrated a magnified view of a cell 102 from sponge 100. As shown, capture probes 180 (the binding agent) are attached or conjugated on the surface of the cell 102. The probes 180 may be DNA or RNA and may be linked to the surface of the cell 102 though a spacer 182. Spacer 182 places the probe 180 at a predetermined distance, depending on the spacer, from the surface of the solid support to prevent streric hindrance between the surface of the solid support and a DNA or RNA analyte. Spacer 182 may also be cleavable as described above. Sponge 100 may then be exposed to a solution containing an analyte 184 as best described above in Steps I to VI of FIG. 6. The sponge may then be washed to remove non-specifically bound contaminants as described above in conjunction with Steps VII to XII of FIG. 6. FIG. 10B shows analyte 184 bound to the capture probe 180. The sponge is then immersed and incubated, as in Steps I to VI in FIG. 6, in a solution containing signal probes 186 having attached thereto a signal element 188 such as a detectable label including a fluorescent label or a catalyst or enzyme capable of producing a detectable signal such as, for example, the enzymes described above including horse radish peroxidase (HRP). The sponge is once again washed with a wash buffer following the steps described above in conjunction with FIG. 6, Steps VII to XII. FIG. 10C depicts signal probes 186 having signal element 188 bound to the analyte 184 that is bound to capture probe 180 on the surface of the cell 102. If the signal element is an enzyme capable of producing a detectable signal such as HRP, the sponge is then immersed in a reagent solution containing an enzyme substrate, as in Steps XIII to XV of FIG. 6. In the case of HRP, the enzyme substrate may be TMB or ABTS. The enzyme substrate then reacts with the signal element 188 producing a detectable signal 190 as illustrated in FIG. 10D. The solution containing the detectable product 190 is then released into an analysis tube as in Steps XVI to XVIII of FIG. 6. The amount of product 190 is then quantitated using an appropriate analytical instrument. If HRP/ABTS is used to produce the detectable signal 190, the resulting product may be quantitated using a UV-Vis Spectrophotometer. Alternatively, if the signal element is a fluorescent label, the signal probe 186 and analyte 184 may be released into solution by heating the sponge in a buffer at 90 degrees C. for approximately 5 minutes. The solution containing the probes may then be released into a collection or analysis vessel such as in Step XVII of FIG. 6. The amount of analyte in the sample may then be quantitated using a fluorimeter by measuring the fluorescence emitted by the signal probes and comparing the signal generated from a known standard.

Amplifying Captured DNA within the Compressible Material

In an alternate embodiment of the method for using the sponge of the present invention for capturing DNA sequences, a portion or all of the sponge 100 as illustrated in FIG. 10B having captured DNA sequences may be processed for amplification of the captured sequences or analyte 184. This may be done by immersing the sponge 100 or part of it in a polymerase chain reaction (PCR) solution in a PCR vial. The PCR solution may include for example, 1X PCR Buffer, 3.0-4.0 mM MgCl2, 0.2 mM dNTPs, 0.2 uM forward and reverse primers, and 0.05 U/ul Taq. The size of the sponge used for the PCR may vary depending on the size of PCR vial used. The immersed sponge may then be run through a pre-determined PCR themocycle method such as, for example, 50 cycles of the following steps: 94 C for 15 seconds to denature the double stranded DNA, 54 C for 15 seconds for annealing and 72 C for 15 seconds for extension. The amplicons may then be gathered by denaturing the DNA by heating the sponge to 94 C then compressing the sponge and collecting the solution expelled from the sponge 100. As would be apparent to one of skill in the art, processing of bound analyte within the sponge 100 is not limited to genetic assays. Any captured analyte including proteins and antibodies may be further processed within the sponge without need for eluting the analyte out of the sponge 100.

Microfiber Solid Support

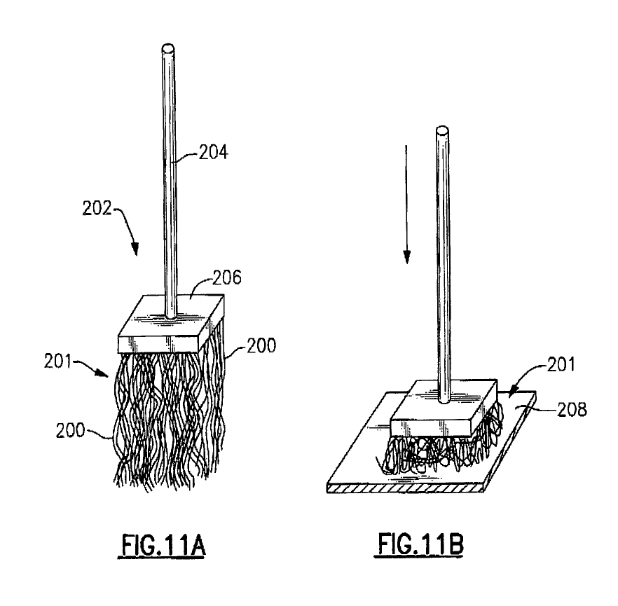

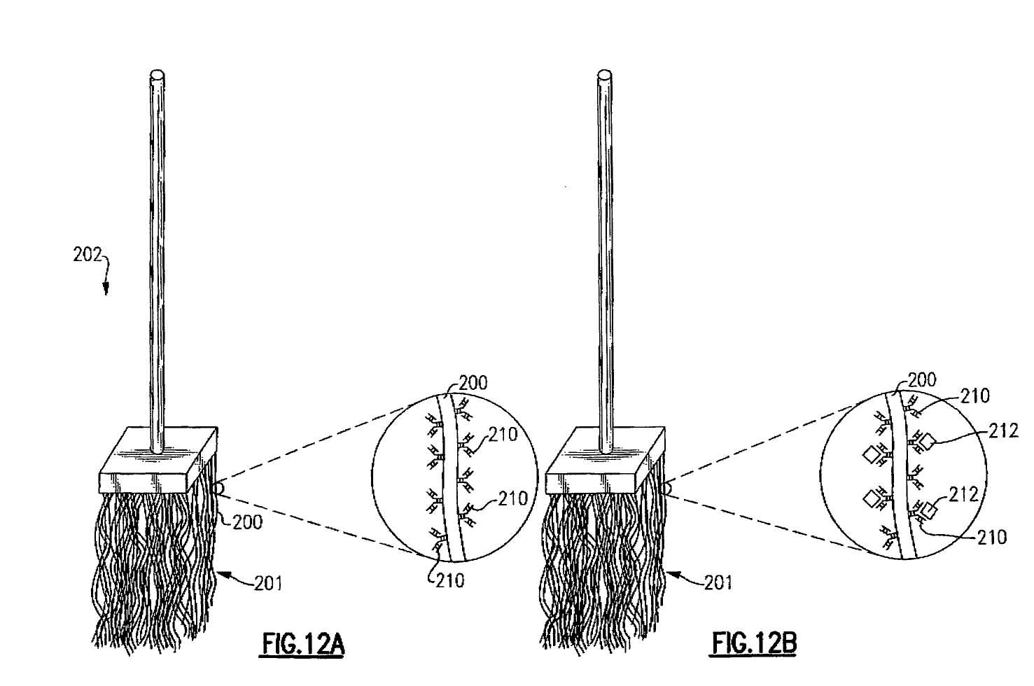

Referring now to FIG. 11A, there is illustrated a perspective view of an alternate embodiment of the present invention using a microfiber material 200 as a solid phase for sample extraction and manipulation. The microfiber material 200 may be formed from natural or synthetic materials including, but not limited to, cotton fibers. The microfiber material may be coated with a hydrophobic or hydrophilic substance. The microfiber material may be bundled together to form a larger fiber such as that used in common cotton maps or cotton brushes. The bundles may be 1 um to 1 mm in diameter. The microfiber bundle is preferably absorbent and allow expulsion of absorbed fluid upon compression thereof, such as, for example, a cotton ball. A cotton ball absorbs liquid when decompressed and releases it when compressed.

With continuing reference to FIG. 11A, a bundle of microfiber material 201 of the present invention may be attached to a plunger 202 having a handle 204 and a base 206 to form a mop-like device. The microfibers 200 are connected at the base 206 of the plunger as illustrated in FIG. 11A. The plunger 202 is used to manipulate the microfiber 200 and aid in ease of its compression and decompression. The microfiber 200 is preferably formed from a material that can be twisted or compressed against a surface to remove liquid. FIG. 11B next shows the microfiber bundle 201 of the mop-like device being compressed on a solid platform 208 using the plunger 202. Just like a household mop, liquid within the microfiber bundle 201 is released upon compression of the microfiber bundle. A main characteristic of the microfiber bundle is its ability to absorban amount of liquid and then expel that liquid upon compression.

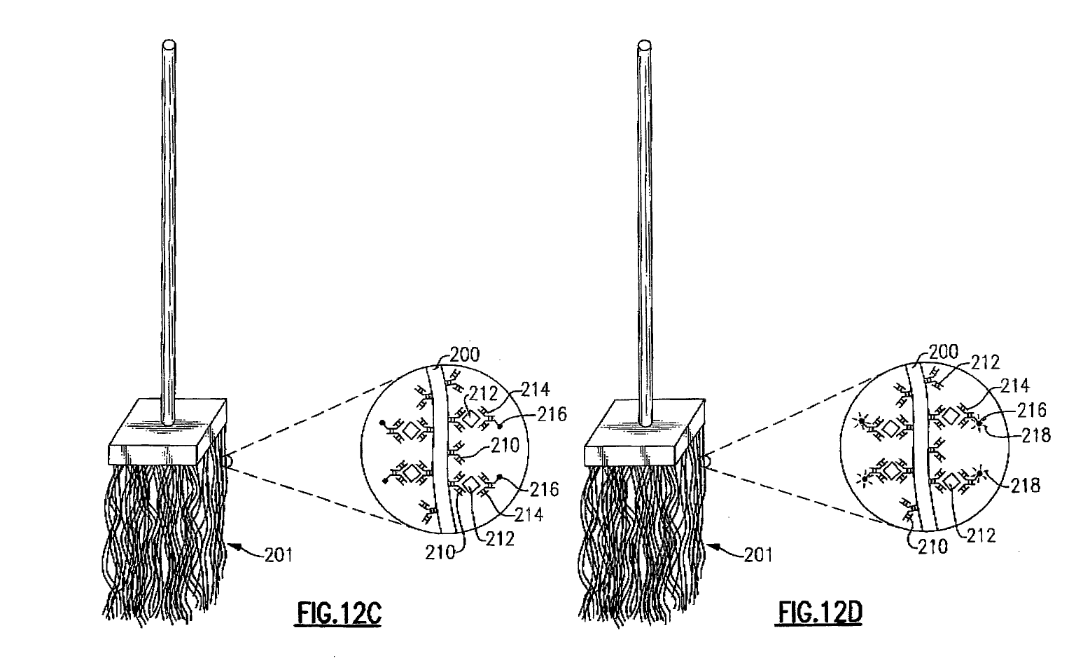

With reference now to FIGS. 12A to 12D, there is shown a method for detecting an analyte using the mop-like device, described above in conjunction with FIGS. 11A and 11B, in an immunoassay or binding assay implementation of the present invention. Binding assays as described above, may use antibodies as binding agents. The assay illustrated in FIGS. 12A to 12D is an antibody-analyte-antibody sandwich assay. As would be apparent to one of skill in the art, the use of the microfiber material of the present invention is not limited to the antibody-analyte-antibody sandwich assay but may be used for all binding assays described above. In FIG. 12A, there is illustrated a magnified view of the microfiber 200. As shown, capture antibodies 210 (the binding agent) are attached or conjugated on the surface of the microfiber 200. Microfiber bundle 201 may then be exposed to a solution containing an analyte as best described above in Steps I to VI of FIG. 6. The bundle 201 may then be washed to remove non-specifically bound contaminants as described above in conjunction with Steps VII to XII of FIG. 6. FIG. 12B shows an analyte 212 bound to the capture agent or antibody 210. The bundle 201 is then immersed and incubated in a solution containing signal agents or signal antibodies having attached thereto a signal element such as a detectable label such as a fluorescent label or a catalyst or enzyme capable of producing a detectable signal such as, for example, the enzymes described above including horse radish peroxidase (HRP); as in Steps I to VI in FIG. 6. The bundle 201 is once again washed with a wash buffer following the steps described above in conjunction with FIG. 6, Steps VII to XII. FIG. 12C depicts signal agents 214 having a signal element 216 bound to the analyte 212. If the signal element 216 is an enzyme capable of producing a detectable signal such as HRP, the bundle 201 is then immersed in a reagent solution containing an enzyme substrate, as in Steps XII to XV of FIG. 6. In the case of HRP, the enzyme substrate may be TMB or ABTS. The enzyme substrate then reacts with the signal element producing a detectable signal 218 as illustrated in FIG. 12D. The solution containing the detectable product 218 is then released into an analysis tube as in Steps XVI to XVIII of FIG. 6. The amount of product 218 is then quantitated using an appropriate analytical instrument. If HRP/ABTS is used to produce the detectable signal 218, the resulting product may be quatitated using a UV-Vis Spectrophotometer.

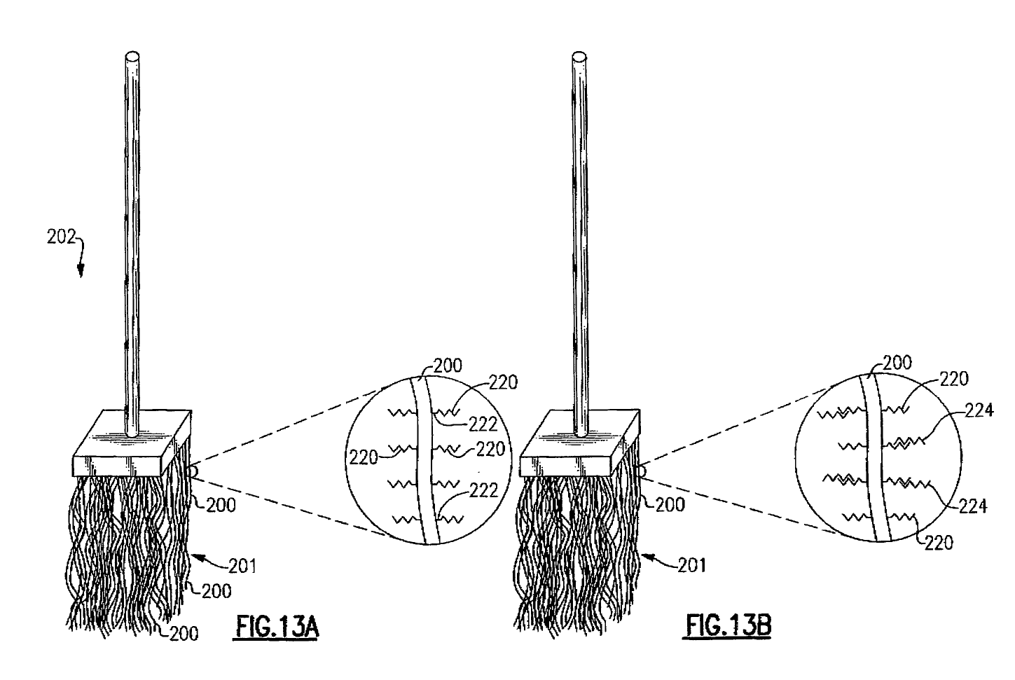

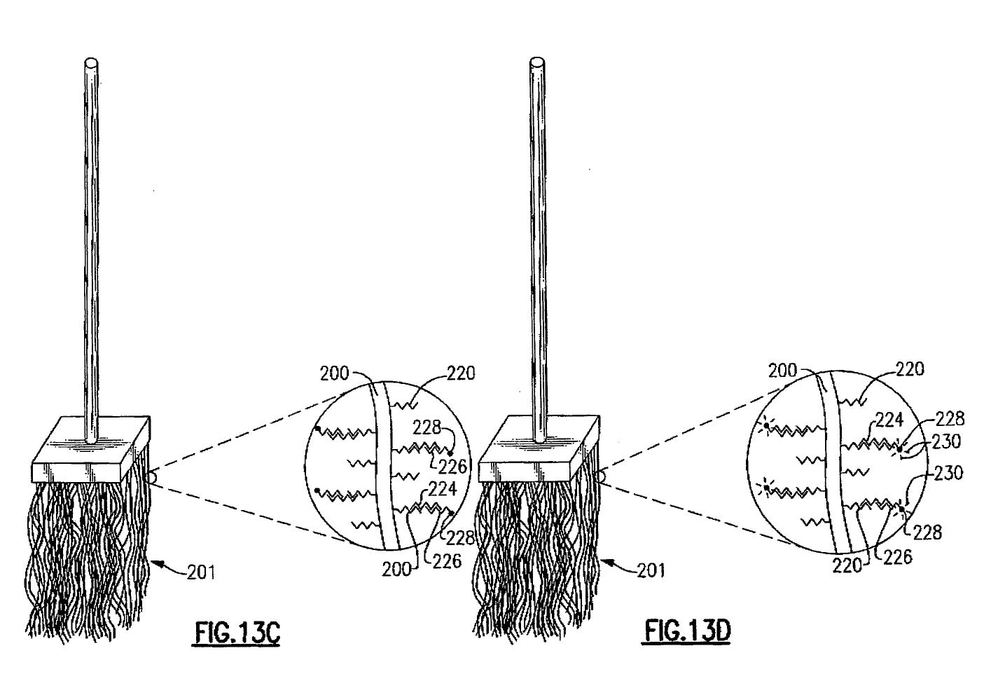

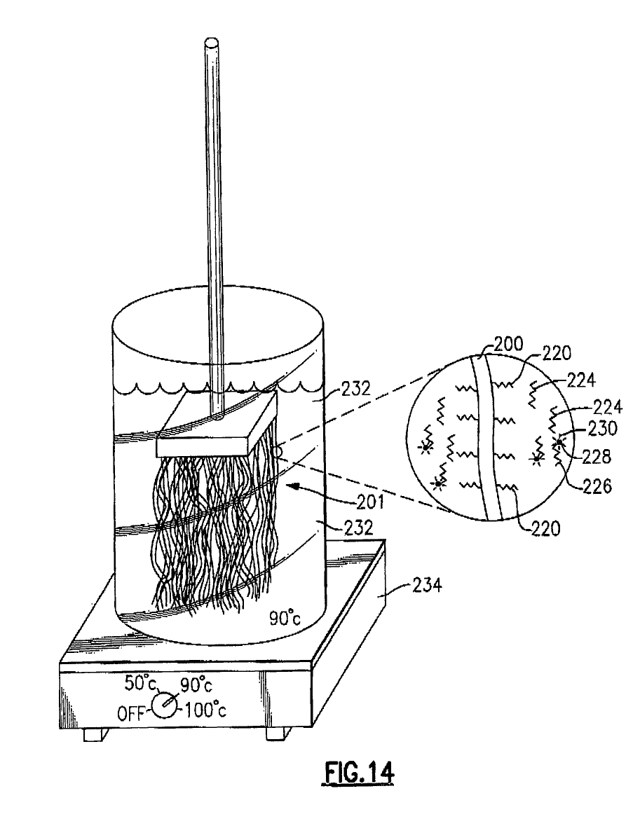

Turning next to FIGS. 13A to 13D there are shown steps for detecting an oligonucleotide sequence of interest in a sample using the compressible microfiber bundle 201 in a genetic assay implementation of the present invention. In FIG. 13A, there is illustrated a magnified view of the microfiber 200. As shown, capture probes 220 (the binding agent) are attached or conjugated on the surface of the microfiber 200. The probes 220 may be DNA or RNA and may be linked to the surface of the microfiber 200 though a spacer 222. Spacer 222 places probe 220 at a predetermined distance, depending on the spacer, from the surface of the solid support to prevent streric hindrance between the surface of the solid support and the DNA or RNA analyte. Spacer 222 may also be cleavable as described above. Bundle 201 may then be exposed to a solution containing an analyte as best described above in Steps I to VI of FIG. 6. The bundle 201 may then be washed to remove non-specifically bound contaminants as described above in conjunction with Steps VII to XII of FIG. 6. FIG. 13B shows an analyte 224 bound to the capture probe 220. The bundle 201 is then immersed and incubated, as in Steps I to VI in FIG. 6, in a solution containing signal probes having attached thereto a signal element such as a detectable label including a fluorescent label or a catalyst or enzyme capable of producing a detectable signal such as, for example, the enzymes described above including horse radish peroxidase (HRP) and Alkaline Phosphatase (AP). The bundle 221 is once again washed with a wash buffer following the steps described above in conjunction with FIG. 6, Steps VII to XII. FIG. 13C depicts signal probes 226 having a signal element 228. Signal probe 226 is bound to the analyte 224 that is bound to capture probe 220 on the surface of the microfiber 200. If the signal element is an enzyme capable of producing a detectable signal such as HRP or AP, the microfiber bundle is then immersed in a reagent solution containing an enzyme substrate, as in Steps XIII to XV. In the case of AP, the enzyme substrate may be pNPP (p-Nitrophenylphosphate). The enzyme substrate then reacts with the signal element 228 producing a detectable signal 230 as illustrated in FIG. 13D. The solution containing the detectable product 230 is then released into an analysis tube as in Steps XVI to XVIII of FIG. 6. The amount of product 230 is then quantitated using an appropriate analytical instrument. If AP/pNPP is used to produce the detectable signal 230, the resulting product may be quantitated using a UV-Vis Spectrophotometer at 405 mm wavelength. Alternatively, if the signal element is a fluorescent label, the signal probe 226 and analyte 224 may be released into solution by heating the microfiber in a buffer heated to 90 degrees C., as illustrated in FIG. 14, for approximately 5 minutes. As shown in FIG. 14, the bundle 221 is immersed in an elution buffer 232 heated to 90 degrees C. using a hotplate 234. The solution or buffer 232 containing the probes may then be analyzed to determine the amount of analyte in the sample using a fluorimeter by measuring the fluorescence emitted by the signal probes and comparing the signal generated to a known standard.

Experimental Details

While this invention has been described in detail with reference to the drawing figures, certain examples and further details of the invention are presented below. These examples are provided by way of illustration, and are not intended to be limiting of the present invention.

### EXAMPLE 1

### DNA Purification from Cell Lysate using Silica Gel Functionalized Sponge

a. Activation of Sponge.

A PVA (polyvynylalcohol) sponge (UltraPure PVA, Shima, San Jose, Calif.) is cut into sheets that are 6×6 inches wide and 1 cm thick. The sponge is activated by reaction the hydroxyl groups of the PVA with 2-CYANOETHYLTRIETHOXYSILANE. The sponge is submerged in a 200 ml of 1 mM 2-CYANOETHYLTRIETHOXYSILANE in ethyl acetate for approximately 5-10 minutes at 37 C. Mixing is effected by compressing and decompressing the sponge every 0.5 minutes. The sponge is rinsed by immersion and repeated compression/decompression in 100% ethyl acetate for 3 minutes. This is done 3 times with fresh solution.

b. Attachment of Silica Particles to Activated Sponge.

Silica gel particles are bound to the activated sponge of Part A. A aqueous silica gel slurry is made by adding 3 g of silica gel (Aldrich chemical, TLC grade avg. particle size 2-25 um), to 1000 ml of water with pH adjusted to appropriate levels using NaOH or HCl.

The sponge is placed on a grid and the slurry is flowed through the sponge and re-circulated to maintain even flow through the sponge for 20 minutes. The sponge is then rinsed of unbound particles by immersion and repeated compression/decompression in 500 ml DI water, 5 successive immersions in fresh water with constant compression/decompression for 3 minutes. The sponge is dried in an oven at 50 C overnight.

c. Cutting of Sponge and Mounting of Manipulator.

The sponge is cut into pieces 7 mm×7 mm×10 mm. An individual piece is mounted to a 7×7 mm flat surface with a rod extending a few inches normal to the flat surface on the side opposite the sponge. The rod is for handling during extraction. Adhesion to the sponge is carried out by first applying a small amount (50 uL) of heat activated adhesive evenly to one surface of the flat then the flat is pushed against the sponge and held in place for 5 minutes.

d. DNA/RNA Extraction/Purification.

100 ul of cell lysate solution is placed in a vessel (see FIG. 6). The silica activated sponge is immersed in the solution and compressed/decompressed at a rate of 1 up/down cycle/second for 20 seconds. The excess solution is removed from the sponge by compression against the slanted surface in the middle of the vessel. The sponge is rinsed in a fresh vessel containing 50 mM Phosphate buffered Saline solution and 0.1% tween 20 by compressed decompressed at a rate of 1 up/down cycle/second for 20 seconds. The excess rinse solution is removed from the sponge by compression against the slanted surface in the middle of the vessel. This rinse is repeated once more in a fresh rinse solution. The DNA/RNA is extracted by immersing the sponge into 100 ul of De-Ionized water for 2 minutes with compressed/decompressed at a rate of 1 up/down cycle/second. The sponge is then compressed against the slanted surface in the middle of the vessel to remove the solution with the purified DNA/RNA.

### EXAMPLE 2

### DNA Purification from Cell Lysate using Ion-exchange Sponge

a. Activation of Sponge.

A PVA (polyvynylalcohol) sponge (UltraPure PVA, Shima, San Jose, Calif.) is cut into sheets that are 6×6 inches wide and 1 cm thick. The sponge is activated by reaction the hydroxyl groups of the PVA with Carbonyldiimidazole (CDI). The sponge is submerged in a 200 ml of CDI in ethyl acetate for 15 minutes at room temperature. Mixing is effected by compressing the sponge every minute. The sponge is rinsed by immersion and repeated compression/decompression in 100% ethyl acetate for 3 minutes. This is done 3 times with fresh solution.

b. Attachment of Diethyl Amino Functionality for Anion Exchange Capability to Activated Sponge.

Diethyl amino groups are bound to the activated sponge of part a. The activate sponge is immersed in 300 ml of 3-(Dietyhylamino)propylamine (Aldrich chemical, 5 mM in ethyl acetate) for 15 minutes at 25 C. Mixing is effected by compressing the sponge every minute. The sponge is rinsed by immersion and repeated compression decompression in 100% ethyl acetate for 3 minutes. This is done 3 times with fresh solution. The sponge is dried in an oven at 50 C for overnight.

c. Cutting of Sponge and Mounting of Manipulator.

The sponge is cut into pieces 7 mm×7 mm×10 mm. An individual piece is mounted to a 7×7 mm flat surface with a rod extending a few inches normal to the flat surface on the side opposite the sponge. The rod is for handling during extraction. Adhesion to the sponge is carried out by first applying a small amount (50 uL) of heat activated adhesive evenly to one surface of the flat then the flat is pushed against the sponge and held in place for 5 minutes.

d. DNA/RNA Extraction/Purification.

100 ul of cell lysate solution is placed in a vessel (as depicted and described above in conjunction with FIG. 6). The lysate is then desalted. The anion exchange sponge is immersed in the desalted solution and compressed/decompressed at a rate of 1 up/down cycle/second for 20 seconds. The excess solution is removed from the sponge by compression against the slanted surface in the middle of the vessel (Vessel A, FIG. 7). The sponge is rinsed in a fresh vessel containing PBS buffer by compressed/decompressed at a rate of 1 up/down cycle/second for 20 seconds. The excess rinse solution is removed from the sponge by compression against the slanted surface in the middle of the vessel. This rinse is repeated once more in a fresh rinse solution. The DNA/RNA is extracted by immersing the sponge in a high salt buffer for 2 minutes with compressed/decompressed at a rate of 1 up/down cycle/second. The sponge is then compressed against the slanted surface in the middle of the vessel to remove the solution with the purified DNA/RNA. DNA/RNA can be further purified from buffer solution by addition of ethanol and precipitation/centrifugation.

Concluding Summary

All patents, provisional applications, patent applications, and other publications mentioned, referenced, or cited in this specification are incorporated herein by reference in their entireties.

While this invention has been described in detail with reference to certain preferred embodiments, it should be appreciated that the present invention is not limited to those precise embodiments. Rather, in view of the present disclosure that describes the current best mode for practicing the invention, many modifications and variations would present themselves to those of skill in the art without departing from the scope and spirit of this invention. The scope of the invention is, therefore, indicated by the following claims rather than by the foregoing description. All changes, modifications, and variations coming within the meaning and range of equivalency of the claims are to be considered within their scope.

Furthermore, those skilled in the art will recognize, or be able to ascertain, using no more than routine experimentation, many equivalents to the specific embodiments of the invention described herein. Such equivalents are also intended to be encompassed by the following claims.

1. A sample processing device comprising:
a compressible material having interconnected open cells;
one or more capture agents bound to surface of said interconnected open cells;
a plunger attached to said compressible material; and
one or more vessels for containing fluids.

2. The device according to claim 1 wherein said compressible material is formed from PVA.

3. The device according to claim 2 wherein said one or more capture agents is selected from the group comprising antibodies, antigens, DNA, RNA, and binding proteins.

4. A method of using the device of claim 3 comprising the steps of:
placing a sample containing an analyte into said one or more vessels;
immersing said compressible material into said sample in said one or more vessels;
allowing said sample to be absorbed into said compressible material;
incubating said sample in said compressible material to allow binding of said analyte to said one or more capture agents;
washing said compressible material by immersing said compressible material in a wash buffer;
compressing and decompressing said compressible material in said wash buffer to facilitate removal of unwanted substances;
eluting out analyte bound to said one or more capture agents in said compressible material by immersing said compressible material in an elution buffer capable of disrupting bonds between said one or more capture agents and said analyte;
compressing and decompressing said compressible material in said elution buffer to facilitate elution of said analyte; and
collecting said elution buffer containing said analyte.

5. A method of using a PVA sponge having open interconnected pores for isolating a sample, said method of using comprising the steps of:
attaching one or more capture agents on surface of said PVA sponge;
placing a sample containing an analyte into a sample container;
immersing said PVA sponge into said sample in said sample container;
allowing said sample to be absorbed into said PVA sponge;
incubating said sample in said PVA sponge to allow binding of said analyte to said one or more capture agents;
washing said PVA sponge by immersing said PVA sponge in a wash buffer;
compressing and decompressing said PVA sponge in said wash buffer to facilitate removal of unwanted substances;
eluting out analyte bound to said one or more capture agents in said PVA sponge by immersing said PVA sponge in an elution buffer capable of disrupting bonds between said one or more capture agents and said analyte;
compressing and decompressing said PVA sponge in said elution buffer to facilitate elution of said analyte; and
collecting said elution buffer containing said analyte.

6. The method according to claim 5 further comprising the step of compressing and decompressing the PVA sponge in said sample containing said analyte to enhance binding kinetics between said analyte and said one or more capture agents.

7. A sample processing device comprising:
an absorbent material formed from microfibers;
a handle connected to said absorbent material;
one or more capture agents bound to surface of said microfibers; and
one or more vessels for containing fluids.

8. The device according to claim 7 wherein said microfibers are formed from cotton fibers.

9. The device according to claim 8 wherein said one or more capture agents is selected from the group comprising antibodies, antigens, DNA, RNA and binding proteins.

10. A method of using the device of claim 9 comprising the steps of:
placing a sample containing an analyte into said one or more vessels;
immersing said absorbent material into said sample in said one or more vessels;
allowing said absorbent material to absorb said sample;
incubating said sample in said absorbent material to allow binding of said analyte to said one or more capture agents;
washing said absorbent material by immersing said absorbent material in a wash buffer;
compressing and decompressing said absorbent material in said wash buffer to facilitate removal of unwanted substances;
eluting out analyte bound to said one or more capture agents on said microfibers by immersing said absorbent material in an elution buffer capable of disrupting bonds between said one or more capture agents and said analyte;
compressing and decompressing said absorbent material in said elution buffer to facilitate elution of said analyte; and
collecting said elution buffer containing said analyte.

11. A method of using a cotton ball formed from cotton fibers for isolating a sample, said method of using comprising the steps of:
attaching one or more capture agents on surface of said cotton fibers;
placing a sample containing an analyte into a sample container;
immersing said cotton ball into said sample in said sample container;
allowing said cotton ball to absorb said sample;
incubating said sample in said cotton ball to allow binding of said analyte to said one or more capture agents;
washing said cotton ball by immersing said cotton ball in a wash buffer;
compressing and decompressing said cotton ball in said wash buffer to facilitate removal of unwanted substances;
eluting out analyte bound to said one or more capture agents on said cotton fibers by immersing said cotton ball in an elution buffer capable of disrupting bonds between said one or more capture agents and said analyte;
compressing and decompressing said cotton ball in said elution buffer to facilitate elution of said analyte; and
collecting said elution buffer containing said analyte.

12. The method according to claim 11 further comprising the step of compressing and decompressing said cotton ball in said sample containing said analyte to enhance binding kinetics between said analyte and said one or more capture agents.

13. A method of using the device of claim 3 comprising the steps of:
placing a sample containing a DNA analyte into said one or more vessels;
immersing said compressible material into said sample in said one or more vessels;
allowing said sample to be absorbed into said compressible material;
incubating said sample in said compressible material to allow binding of said DNA analyte to said one or more DNA capture agents;
washing said compressible material by immersing said compressible material in a wash buffer;
compressing and decompressing said compressible material in said wash buffer to facilitate removal of unwanted substances;
placing said compressible material having captured DNA analyte into a PCR vial;
adding a pre-determined volume of PCR solution;
placing the PCR vial onto a thermocycler;
running a pre-determined PCR themocycle to amplify said captured DNA analyte to generate an amplicon; and
collecting said amplicon.

14. The method according to claim 14 further including the step of heating said compressible material to 95 degrees C. for 30 seconds prior to the collecting step.
